(19) United States
(12) Patent Application Publication
(10) Pub. No.: US 2021/0378277 A1
(43) Pub. Date: Dec. 9, 2021

(54) COMPOSITION AND METHOD TO PREVENT PATHOGEN TRANSMISSION THROUGH ALTERING SALIVA

(71) Applicant: University of Central Florida Research Foundation, Inc., Orlando, FL (US)

(72) Inventors: Michael Kinzel, Orlando, FL (US); Kareem A. Ahmed, Orlando, FL (US); Jonathan Reyes, Orlando, FL (US); Douglas H. Fontes, Orlando, FL (US); Michelle Otero, Orlando, FL (US)

(21) Appl. No.: 17/303,736

(22) Filed: Jun. 7, 2021

Related U.S. Application Data
(60) Provisional application No. 63/035,407, filed on Jun. 5, 2020.

(51) Int. Cl. A23L 29/238

(52) U.S. Cl. CPC A23L 29/238

(57) ABSTRACT

Airborne transmitted pathogens, including severe acute respiratory coronavirus 2 (SARS-CoV-2) and similar infectious diseases, often spread through close contact between humans. One common form of transmission is that of saliva droplets that suspend in the air and are subsequently inhaled by another human, leading to pathogen transmission. The application is based on formulated confections (such as lozenges, cough drops, gum, candy, buccal films, and other consumable products) and/or medicinal drugs that alter fluid properties and/or reduce quantity of the saliva. As a result, the modified fluid properties of the saliva mitigates droplet breakup, resulting in larger droplets that travel shorter distances before quickly falling to a ground surface. The saliva reduction reduces the amount of saliva. As a result, the formulated confections reduce the prevalence of airborne transmitted pathogens (such as SARS-CoV-2) with a lesser/thickening saliva.

### CROSS-REFERENCE TO RELATED APPLICATIONS

This nonprovisional application is a continuation of and claims priority to provisional application No. 63/035,407, entitled “Composition and method to prevent pathogen transmission through altering saliva,” filed Jun. 5, 2020, by the same inventors.

### BACKGROUND OF THE INVENTION

### 1. Field of the Invention

This invention relates, generally, to saliva droplets. More specifically, it relates to compositions and methods for increasing saliva droplet size, density and weight, as well as decreasing saliva droplet production, to minimize droplet presence in the area surrounding a human and maximize the droplet's size, such that exhaled droplets reach a ground surface at an increased rate. As such, the invention relates to the prevention or reduction in pathogen transmissions caused by exhaled saliva droplets by reducing the number of droplets suspended above-ground in the area surrounding a human.

### 2. Brief Description of the Prior Art

Airborne transmitted pathogens, including severe acute respiratory coronavirus 2 (SARS-CoV-2) and similar infectious diseases, often spread through close contact between humans. Specifically, airborne transmission paths associated with natural human respiratory functions, such as sneezing, coughing, speaking, and breathing, are driven by droplets that carry the pathogens. These pathogen-laden droplets are ejected from the host and, depending on the droplet size, can take various paths for transmission. For example, larger droplets tend to fall quickly toward a ground surface due to gravity and the increased weight of the droplets. However, smaller droplets remain suspended in the air as an aerosol and are prone to intake into ventilation systems.

Such aerosol droplets have been particularly dangerous during the SARS-CoV-2-related pandemic, which has led to large-scale infections, death, health-system overloads, and severe economic damage. These aerosols involve droplets are inhaled by uninfected hosts, and exposure is unexpected. The uninfected hosts may become asymptomatic carriers of the virus, and subsequent aerosol droplets risk infection to greater numbers of potential hosts, resulting in exponential virus transmission. However, larger droplets that are not easily inhaled by new hosts pose a lesser risk, since the droplets tend to reach a ground surface faster than aerosol droplets.

Social distancing efforts and the use of face masks aim to reduce transmission from droplets. However, such efforts are not infallible and rely on widespread implementations, and deviations from such efforts by small groups can place entire communities at risk of infection. Accordingly, what is needed is a composition and method for reducing the transmissibility of airborne pathogens by altering fluid properties from the source to prevent/reduce fine-scale droplets formation from human respiratory functions. However, in view of the art considered as a whole at the time the present invention was made, it was not obvious to those of ordinary skill in the field of this invention how the shortcomings of the prior art could be overcome.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of the invention, Applicant in no way disclaims these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for a composition and a method for increasing saliva droplet sizes to reduce the transmissibility of airborne pathogens is now met by a new, useful, and nonobvious invention.

The novel composition to increase saliva density, viscosity, and/or surface tension is configured to reduce a transmissibility of airborne pathogens. The composition includes a thickening agent selected from the group consisting of cornstarch, xanthan gum, glucomannan powder, gelatin, peanut flour, agar-agar, arrowroot flour, algin, carrageenan, acacin, gum tragacanth, pectin, and combinations thereof. The thickening agent is present within the composition in an amount of between 2% and 40% (w/w). The composition also includes a saliva-reduction agent selected from the group consisting of a starch beta blocker, a natural beta blocker, an oil, a natural extract, lemongrass oil, ginger extract, cinnamaldehyde, vanillin, peppermint, and combinations thereof. The saliva-reduction agent is present within the composition in an amount of between 1% and 25% (w/w). A bonding agent is present in the composition, such as being present within the composition in an amount of between 20% and 95% (w/w). A food grade carrier encapsulates the thickening agent, the saliva-reduction agent, and the bonding agent therein, with the food grade carrier being selected from the group consisting of a cough drop, a dissolvable strip, a gum, and an edible food item. In an embodiment, the edible food item is a chocolate-based food item.

An embodiment of the composition is a synergistic composition that includes the thickening agent, the saliva-reduction agent, the bonding agent, and the food grade carrier. The thickening agent is configured to interact with a salivary gland of a user to increase at least one of a density, a viscosity, and a surface tension of saliva produced by the salivary gland thereby thickening the produced saliva. The thickening agent is configured to reduce the transmissibility of airborne pathogens due to the thickened saliva produced by the salivary gland, resulting in exhaled saliva droplets translating toward a ground surface at an increased downward vertical velocity and a decreased horizontal velocity compared with unmodified saliva droplets, thereby reducing exhaled saliva droplet transmission. The saliva reduction agent is configured to interact with the salivary gland of the user to decrease saliva production. The saliva reduction agent is configured to reduce the transmissibility of airborne pathogens due to the decreased saliva produced by the salivary gland, resulting in fewer exhaled saliva droplets, thereby reducing exhaled saliva droplet transmission.

An embodiment of the composition includes a palatability agent disposed within the food grade carrier. The palatability agent is selected from the group consisting of honey, cocoa butter, a flavoring oil, a fruit flavoring oil, a fruit flavoring essence, an aldehyde, an ester, and combinations thereof.

An embodiment of the composition includes a nutritional supplement within the food grade carrier. The nutritional supplement is selected from the group consisting of vitamin B, vitamin C, vitamin D3, zinc, N-acetylcysteine, melatonin, and combinations thereof.

A novel method of reducing a transmissibility of airborne pathogens includes a step of combining a synergistic composition including a thickening agent, a saliva-reduction agent, and a bonding agent in a food grade carrier that encapsulates the thickening agent, the saliva-reduction agent, and the bonding agent therein. The synergistic composition is applied to a user, such as by ingestion. The saliva-reduction agent decreases saliva production from a salivary gland of the user. The saliva-reduction agent also reduces a transmissibility of airborne pathogens due to the decreased saliva produced by the salivary gland, resulting in fewer exhaled saliva droplets, thereby reducing exhaled saliva droplet transmission. The thickening agent increases at least one of a density, a viscosity, and a surface tension of saliva produced by the salivary gland of the user, thereby thickening the produced saliva. The thickening agent also reduces the transmissibility of airborne pathogens due to the thickened saliva produced by the salivary gland, resulting in exhaled saliva droplets translating toward a ground surface at an increased downward vertical velocity and a decreased horizontal velocity as compared with unmodified saliva droplets, thereby reducing exhaled saliva droplet transmission. As such, the thickening agent and the saliva-reduction agent act synergistically to reduce saliva production and thicken saliva particles to reduce a suspension time of saliva droplets, thereby synergistically reducing the transmissibility of airborne pathogens.

An object of the invention is to reduce the prevalence of aerosol droplets during natural human respiratory functions, thereby reducing the transmissibility of airborne pathogens.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a fuller understanding of the invention, reference should be made to the following detailed description, taken in connection with the accompanying drawings, in which:

FIG. 1 depicts droplet dispersion (section A and section B) and histograms of droplet sizes (section C and section D) for saliva droplets (section A and section C) and modified saliva droplets (section B and section D).

FIG. 2 depicts instantaneous images of a sneeze taken 93 ms, 279 ms, and 465 ms after the start of a sneezing event for: unmodified saliva (section A); saliva modified with a thickening agent (section B); and unmodified saliva in the presence of a mask on the subject (section C).

FIG. 3 depicts instantaneous velocity contours of the samples of FIG. 2 taken 185 ms after the start of the sneezing event, including velocity contours of unmodified saliva (section A); saliva modified with a thickening agent (section B); and unmodified saliva in the presence of a mask on the subject (section C).

FIG. 4 depicts corrected average particle spatial distribution for the test cases of FIGS. 2-3.

FIG. 5 depicts instantaneous images of a sneeze taken after the start of a sneezing event for: unmodified saliva (section A); unmodified saliva and a surgical mask (section B); saliva modified with a thickening agent (section C); and saliva-reduction agent (section D).

FIG. 6 depicts images and measures of droplet spray content following a human respiratory event, showing pure thickening (section A), pure saliva reduction (section B), and combinations of the thickening and saliva-reduction agents (section C).

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supersedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about.” As used herein, “about” or “approximately” refers to being within an acceptable error range for the particular value as determined by one of ordinary skill in the art, which will depend in part on how the value is measured or determined. As used herein, the term “about” refers to ±10% of the numerical; it should be understood that a numerical including an associated range with a lower boundary of greater than zero must be a non-zero numerical, and the term “about” should be understood to include only non-zero values in such scenarios.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used herein, “subject” is used to describe a human or other animal to whom treatment is administered, including prophylactic treatment with the compositions of the present invention.

As used herein, the term “pharmaceutically acceptable carrier” is used to describe any of the standard pharmaceutically acceptable carriers. The pharmaceutically acceptable carrier can include excipients such as diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils.

Any of the compositions disclosed herein may be administered with or without an excipient. Excipients include, for example, encapsulating materials or additives such as release modifying agents; efficacy enhancing agents; absorption accelerators; antioxidants; binders; buffers; coating agents; pigments; diluents; disintegrating agents; emulsifiers; extenders; fillers; flavoring agents; humectants; lubricants; preservatives; sterilizing agents; solubilizers; wetting agents; and mixtures thereof.

As used herein, “administering” or “administration” refers to the process by which the compositions of the present invention are delivered to a subject for treatment purposes. The compositions of the present invention may be administered orally to the subject in dosage unit formulations containing conventional nontoxic pharmaceutically acceptable carriers, adjuvants, and vehicles as desired. Administration may occur once or multiple times.

The present invention includes compositions direct to disruptions to the airborne transmission pathway by reducing the number of aerosols formed during human respiratory function. In general, droplet initiation involves larger droplets that are ejected from stripping from films. These larger droplets incur flow instabilities that lead to breakup into smaller droplets when exposed to a flow. This breakup is strongly tied to flow speed and fluid properties. The key relevant drivers are: (1) the Weber number, which is the ratio of droplet inertia to surface tension; and (2) the Ohnesorge number, which is the ratio of viscous force to inertial/surface tension. In general, the flow speeds are established from the human respiratory event itself. Hence, a sneeze drives an increased velocity, whereas speaking involves a lower velocity. The fluid properties involve not only the fluid properties of the air, but also those of the salvia/mucus of the host. By altering these parameters, the transmission pathway for airborne pathogens associated with droplet break-up mechanisms can be altered.

Accordingly, the present invention includes compositions designed to increase saliva droplet sizes to thereby decrease droplet suspension time, which in turn decreases airborne pathogen spread. The following non-limiting examples illustrate exemplary compositions and components thereof, including methods of use thereof, in accordance with various embodiments of the disclosure. The examples are merely illustrative and are not intended to limit the disclosure in any way.

### EXAMPLES

Compositions to yield the modified saliva described in the methods below include one or more agents designed to alternatively or in combination thicken and reduce the saliva, with other agents optionally included in the composition to accomplish goals ranging from decreasing salivary gland production to enhancing the composition's palatability. In an embodiment, a synergistic combination of one or more thickening agents with one or more saliva-reduction agents is included within the composition to accomplish dual goals of reducing saliva production while simultaneously thickening the saliva that is produced; as such, upon an expulsion event (such as sneezing, coughing, talking, or otherwise ejecting saliva from a mouth or nostril of a subject), the reduced amount of saliva includes an increased associated vertical velocity in a direction toward a ground surface as compared with unmodified saliva (as will be discussed in the sections below). Such synergism experienced by the composition helps not only reduce the amount of saliva produced by the subject, also to reduce the prevalence of aerosolized droplets disposed above the ground surface, which can be dangerous in the transmission of airborne pathogens.

The compositions described herein include at least one or more thickening agents comprising between about 2% and 40% (w/w) of the composition; one or more saliva-reduction agents comprising between about 1% and 25% (w/w) of the composition; and one or more bonding agents comprising between about 20% and 95% (w/w) of the composition. The composition is incorporated into a pharmaceutically acceptable carrier that is non-toxic and safe for human consumption, such as by ingestion (alternatively referred to as a food-grade carrier).

Thickening agents include, but are not limited to, cornstarch-based agents, xanthan gum, glucomannan powder, gelatin, peanut flour, agar-agar, arrowroot flour, algin, carrageenan, acacin, gum tragacanth, pectin, and combinations thereof.

Saliva-reduction agents include, but are not limited to, beta blockers, such as starch beta blockers (such as those derived from potato and garlic) and natural beta blockers (such as those derived from fish and garlic), natural anticholinergics (such as ginger and capsaicin), oils (such as peppermint, capsicum, and lemongrass), natural extracts (such as ginger and capsicum), cinnamaldehyde, vanillin, peppermint, derivatives thereof, extracts thereof, and combinations thereof.

Bonding agents include, but are not limited to, edible oils (such as nut-derived oils, cocoa butter, seed-derived oils, and peanut oils), chocolate, oligosaccharides (such as sucrose, lactose, and maltose), syrups (such as glucose syrup and high fructose corn syrup), dextrose, dextrins, derivatives thereof, extracts thereof, and combinations thereof.

In addition, embodiments of the compositions described herein can include natural and synthetic palatability agents in the amount of up to about 50% (w/w), including, but not limited to, honey; cocoa butter; flavoring oils such as spearmint, peppermint, cinnamon, wintergreen, olive, almond, and other oils; fruit flavorings including oils and/or essences such as banana, coconut, cherry, grape, orange, citrus, lemon, lime, mango, pineapple, strawberry, apple, melon, raspberry, pear, pomegranate, and similar flavorings; aldehydes; esters; derivatives thereof, extracts thereof; and combinations thereof. In embodiments, vitamins are added to the composition to provide secondary benefits to the user by increasing vitamin intake, such as vitamin B, vitamin C, vitamin D3, zinc, N-acetylcysteine, melatonin, and similar supplements.

In use, the thickening agents in the composition interact with the salivary gland to mix with saliva in a user's mouth, thereby thickening the saliva within the user's mouth. As such, during exhale or during coughing events, the thicker saliva falls to a ground surface as an increased rate as compared with unmodified saliva, as described above. In addition, the saliva-reducing agents, including beta blockers, interact with the salivary gland to suppress saliva production. The synergistic result is not only thicker saliva as a result of the thickening agents, but also decreased saliva production due to the beta blockers, further decreasing the transmissibility of airborne pathogens.

To introduce the agents described above to a salivary gland of a user, the compositions described herein are included in a pharmaceutically acceptable carrier, such as a consumable form of a lozenge, a dissolvable strip, a chewing gum, an edible food item such as chocolate, and other consumable products. As used herein, an edible food item is a non-toxic substance that is digestible by a human. For example, the compositions described herein are introduced to a lozenge by mixing the agents in the composition together, and compacting or heating the mixed agents. Alternative approaches include adding composite layers of ingredients or mixtures of the ingredients. The resulting mixture is then cooled to solidify the mixture, thereby retaining the thickening agents within the mixture. In another embodiment, the compositions described herein include one or more agents mixed together for the purpose of spreading the mixture on a strip, lozenge, or gum carrier. The composition is sprinkled, sprayed, or embedded in pores or microcapsules for ultimate consumption after a curing process. Alternative embodiments include disposing a layer of the compositions described herein between layers of dissolvable films for introduction to a user's mouth. In addition, the compositions described herein can be mixed and heated into a liquid for use in a dropper or an edible vial by a user.

Experimental Methods

In numerical simulations, two fluids were considered to evaluate the liquid property effect on droplet dispersion in a sneeze condition. The first fluid is saliva, represented by water properties (ρ=997.6 kg/m³, μ=8.887×10⁻⁴ Pa·s, and σ=0.072 N/m). The second fluid is modified saliva (ρ=1197.07 kg/m³, μ=1.155×10⁻³ Pa·s, and σ=0.108 N/m), which is a denser fluid than the first fluid (saliva). For the simulations, the two-phase flow was solved in a Euler-Lagrange approach, in which the gas flow is solved as a continuous phase and the liquid droplets are solved as discrete particles in the Lagrangian approach. The mass and momentum balance equations for the continuous phase were discretized through the Finite Volume Method with temporal and spatial interpolations of first and second orders, respectively. A one-way coupling interaction is considered between phases and droplet breakup is accounted via Taylor Analogy Breakup (TAB) method. An inlet condition is imposed at the throat normal surface of the human body model. At this boundary condition, the air is injected in the numerical domain at 40 m/s with a duration of 0.01 second. The droplets are injected with normal size distribution at the inlet condition in rest condition, being accelerated by the airflow.

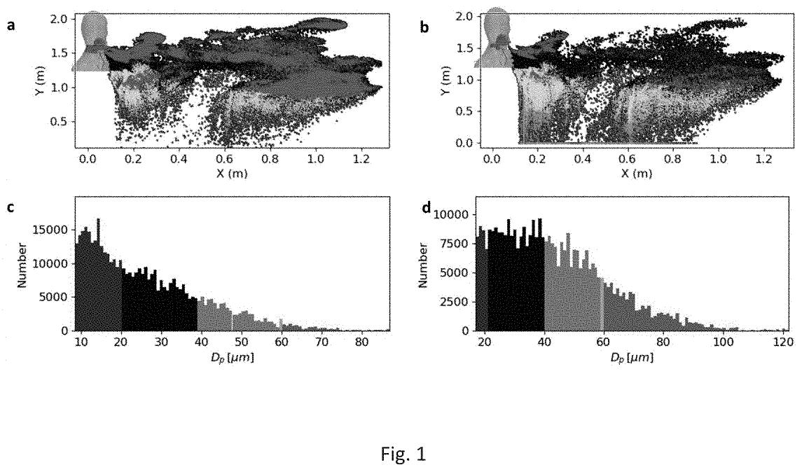

FIG. 1 depicts the suspension of droplets after the sneeze for both saliva (section A of FIG. 1) and modified saliva (section B of FIG. 1). The different size distribution provided from the experiments mimics the spray formation from the liquid film inside the mouth. The further breakup of droplets is accounted for by the TAB model. The droplets of modified saliva remain suspended for less time than those of saliva, which is due to the fact the modified saliva has a greater density. Also, the higher surface tension of the modified saliva reduces the number of droplet breakup, which results in a reduced number of smaller droplets compared to the saliva condition. The occurrence of droplets in the height range of 1.2-1.8 meters (denoted by the yellow bar in sections C and D of FIG. 1, with section C showing droplet sizes for saliva, and section D showing droplet sizes for modified saliva) at 2 seconds after the sneeze is reduced by approximately 25% for the modified saliva compared to the saliva.

A high-speed camera and six light sources are used to record a subject sneezing. The images are captured at 5,400 frames per second and have a spatial resolution of 1.5 mm/pixel for a total span and height of 1523×1523 mm (5×5 ft.). Each light source consists of a 750-watt halogen bulb emitting light from 600 to 1000 nm with a peak at 750 nm. Three light sources are disposed above the region of interest, with the lights being pointed down; three additional lights are disposed below the region of interest and are pointed up. Using the aforementioned light set-up allows for sufficient light scatter of the particulates exiting the subject's nose and mouth, as well as even illumination across the recording region of interest (±7 of an intensity count).

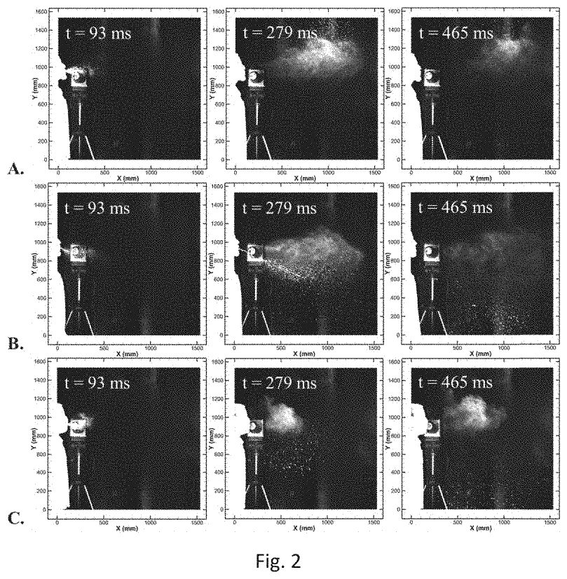

The subject holds a trigger switch that is pressed just before the start of a sneeze to ensure that the full event is captured. Each test case is repeated four times. The subject's spatial location is marked with respect to the recording equipment to ensure consistency in location across test cases. The location of the subject's head can deviate by 100 mm (4 in.) horizontally and vertically at the start of the event and can move up to 205 mm (8 in.) during the event. The angle of the subject's head at the start of the event can deviate by 25 degrees between cases and can deviate as much as 6 degrees during the event. These discrepancies are accounted for by correcting the position (by combinations of translation and rotation) in each image back to an origin and orientation, so that each test case can be analytically compared. The temperature in the room is maintained at 22° C. (72° F.) for each test. In the experiment, three test cases are considered: a sneeze including saliva (shown in section A of FIG. 2); a sneeze including 0.6 mL (⅛ tsp.) of a thickening composition mixed with saliva, such as a cornstarch-based composition mixed with saliva (shown in section B of FIG. 2); and a sneeze in which the subject's nose and mouth are obscured by a mask (mean pore size of 15 microns, 3 layers) (shown in section C of FIG. 2).

FIG. 2 shows instantaneous images taken 93 ms, 279 ms, and 465 ms after the start of the event. In section A of FIG. 2, including unmodified saliva, a dense cloud of floating saliva particulates is seen 900 mm (3 ft.) downstream of the subject. The cloud is approximately 500 mm (1.6 ft.) in size. A similar cloud is apparent in the mask test case (shown in section C of FIG. 2), but the cloud is much closer to the subject as compared to that of unmodified saliva without a mask, located approximately 350 mm (1.1 ft.) downstream of the subject; in addition, the cloud is less dense than that of the case of unmodified saliva without a mask, approximately 400 mm (1.3 ft.) in size. However, such a dense cloud is not seen in the modified saliva including the thickening composition (shown in section B of FIG. 2). Moreover, the modified saliva case has noticeably larger particles in comparison to the unmodified saliva cases, both with and without the mask; in addition, the larger particles are much closer to the ground surface in the modified saliva case.

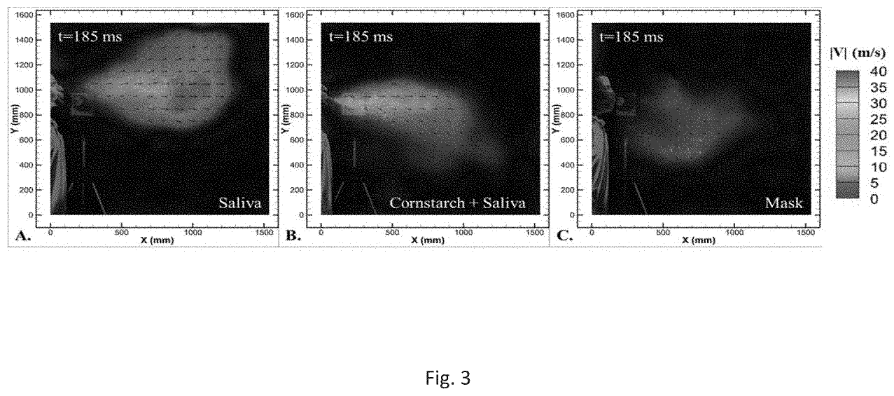

In addition, as shown in FIG. 3, the unmodified saliva test case includes particulate exits from the subject the fastest with horizontal speeds of 30 m/s on average, and a maximum of 42 m/s (section A of FIG. 3). The modified saliva case has an associated average horizontal speed of 23 m/s (34 m/s maximum) (section B of FIG. 3), and the mask case has an associated average horizontal speed of 10 m/s (14 m/s maximum) (section C of FIG. 3). Referring to the vertical velocity of the droplets, the modified saliva case includes the greatest average vertical droplet fall speed of 12 m/s and a maximum of 25 m/s. The mask test case is the second fastest, with an average fall speed of 10 m/s (maximum of 15 m/s). The unmodified saliva test case includes the slowest falling droplet speeds, averaging 6 m/s with a maximum of 10 m/s.

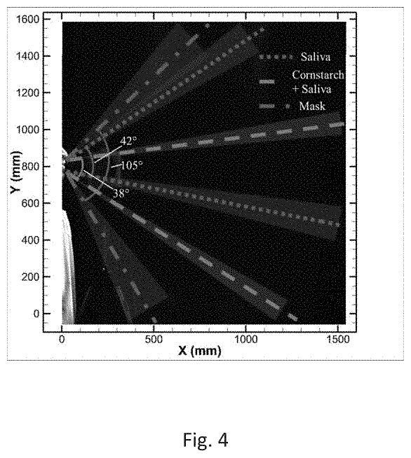

FIG. 4 depicts the corrected average particle spatial distribution from each test case. Each image in the recording is corrected before any calculations are performed by displacing and rotating the image back to a common origin utilizing the mouth of the subject as the center point. The spatial distribution of particles is found by calculating the mean of the event across the multiple tests for a given case. An outer boundary is calculated utilizing a threshold tracing algorithm. A line is drawn from the boundaries and the angle between the lines is calculated. The average area of particle distribution displayed in FIG. 4 shows the mask case having the widest spread at 120 degrees, with the modified saliva case and the unmodified saliva cases having similar spreads of about 40 degrees. However, the trajectory of the particles between the modified saliva case and the unmodified saliva case is different, with the unmodified saliva case being about 5 times higher with respect to a ground surface than the modified saliva case. Moreover, the case including mask particulates are 7 times higher with respect to the ground surface than the modified saliva case. Accordingly, the modified saliva case is found to be associated with droplet particles with a smaller angular spread than distributions associated with mask cases, and with droplet particles disposed closer to the ground surface as compared with unmodified saliva droplets and masked saliva droplets.

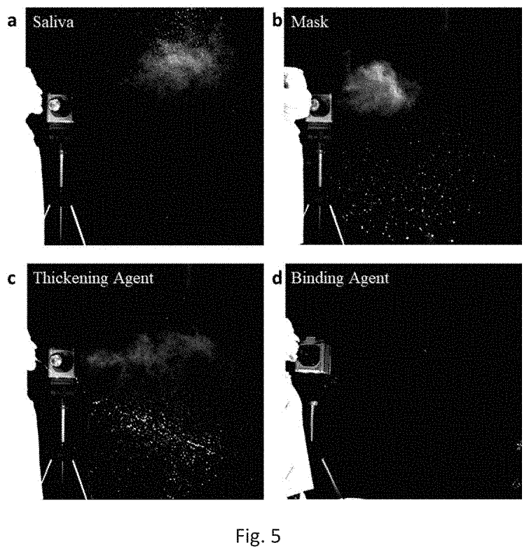

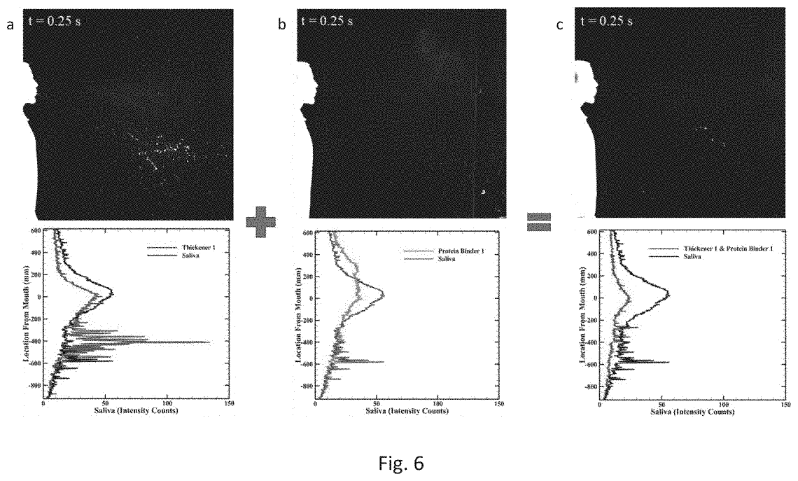

FIGS. 5-6 depict instantaneous images of a sneeze taken after the start of a sneezing event. Specifically, section A of FIG. 5 depicts an image for unmodified saliva; section B of FIG. 5 depicts an image for unmodified saliva and a surgical mask; section C of FIG. 5 depicts an image for saliva modified with a thickening agent; and section D of FIG. 5 depicts an image for saliva modified with a saliva-reduction agent. As shown in FIG. 5, the use of a thickening agent and/or a saliva-reduction agent directs particles downward toward a ground surface and reduces the number of particles expelled during a sneezing event.

Similarly, FIG. 6 depicts images and measurements of modified droplet spray content following a human respiratory event, with the images and measurements taken 0.25 s post-event. Specifically, section A of FIG. 6 depicts an example of a thickening agent used alone to modify saliva; section B of FIG. 6 depicts an example of using a saliva-reduction agent alone to modify saliva; and section C of FIG. 6 depicts an example of using a combination of a thickening agent and a saliva-reduction agent to modify saliva. As shown in FIG. 6, the combination of the thickening agent and the saliva-reduction agent results in decreased saliva droplets in the air, as well as a distribution of droplets proximate to a ground surface, as compared with unmodified saliva.

### Prophetic Example 1

A 40-year-old patient presents with mucoid congestion of the nasal passage, as well as a cough. The patient experiences coughing and sneezing events resulting in the ejection of mucous particles from the patient's nose and mouth into the surrounding area. The patient does not wear a face covering during the coughing and sneezing events, leaving the patient's nose and mouth exposed to the surrounding area. The coughing and sneezing events result in aerosolized droplets into the surrounding area. The patient receives and consumes a composition includes 30% (w/w) of a thickening agent, 25% (w/w) of a saliva-reduction agent, and 45% (w/w) of a bonding agent. After consuming the composition, the patient experiences a reduction in the production of saliva, as well as an increase in a density of the saliva that is produced. Upon a subsequent coughing event and a subsequent sneezing event, after consuming the composition, the patient expels saliva droplets into the surrounding area that include a weight greater than the aerosolized droplets pre-consumption. The heavier saliva droplets fall to a ground surface at an increased rate of acceleration as compared with the saliva prior to consuming the composition.

### Prophetic Example 2

A 40-year-old patient presents with no mucoid congestion and experiences infrequent coughing and sneezing events in the recent history of the patient. The patient does not wear a face covering during the coughing and sneezing events, leaving the patient's nose and mouth exposed to the surrounding area. The patient receives and consumes a composition includes 30% (w/w) of a thickening agent, 25% (w/w) of a saliva-reduction agent, and 45% (w/w) of a bonding agent. After consuming the composition, the patient experiences a reduction in the production of saliva, as well as an increase in a density of the saliva that is produced. Upon a subsequent coughing event and a subsequent sneezing event, after consuming the composition, the patient expels saliva droplets into the surrounding area that include a weight greater than the aerosolized droplets pre-consumption. The heavier saliva droplets fall to a ground surface at an increased rate of acceleration as compared with the saliva prior to consuming the composition.

### Prophetic Example 3

A 40-year-old patient presents with mucoid congestion of the nasal passage, as well as a cough. The patient experiences coughing and sneezing events resulting in the ejection of mucous particles from the patient's nose and mouth into the surrounding area. The patient wears a face covering during the coughing and sneezing events, such that the patient's nose and mouth are covered during the coughing and sneezing events. The coughing and sneezing events result in aerosolized droplets into the surrounding area, which are concentrating in the area immediately surrounding the patient due to the face covering. The patient receives and consumes a composition includes 30% (w/w) of a thickening agent, 25% (w/w) of a saliva-reduction agent, and 45% (w/w) of a bonding agent. After consuming the composition, the patient experiences a reduction in the production of saliva, as well as an increase in a density of the saliva that is produced. Upon a subsequent coughing event and a subsequent sneezing event, after consuming the composition, the patient expels saliva droplets into the surrounding area that include a weight greater than the aerosolized droplets pre-consumption. The heavier saliva droplets fall to a ground surface at an increased rate of acceleration as compared with the saliva prior to consuming the composition.

### Prophetic Example 4

A 40-year-old patient presents with mucoid congestion of the nasal passage, as well as a cough. The patient experiences coughing and sneezing events resulting in the ejection of mucous particles from the patient's nose and mouth into the surrounding area. The patient does not wear a face covering during the coughing and sneezing events, leaving the patient's nose and mouth exposed to the surrounding area. The coughing and sneezing events result in aerosolized droplets into the surrounding area. The patient receives and consumes a composition includes 2% (w/w) of a thickening agent and 5% (w/w) of a saliva-reduction agent. After consuming the composition, the patient experiences a reduction in the production of saliva, as well as an increase in a density of the saliva that is produced. Upon a subsequent coughing event and a subsequent sneezing event, after consuming the composition, the patient expels saliva droplets into the surrounding area that include a weight greater than the aerosolized droplets pre-consumption. The heavier saliva droplets fall to a ground surface at an increased rate of acceleration as compared with the saliva prior to consuming the composition.

### Prophetic Example 5

A 40-year-old patient presents with no mucoid congestion and experiences infrequent coughing and sneezing events in the recent history of the patient. The patient experiences coughing and sneezing events resulting in the ejection of mucous particles from the patient's nose and mouth into the surrounding area. The patient does not wear a face covering during the coughing and sneezing events, leaving the patient's nose and mouth exposed to the surrounding area. The coughing and sneezing events result in aerosolized droplets into the surrounding area. The patient receives and consumes a composition includes 2% (w/w) of a thickening agent and 5% (w/w) of a saliva-reduction agent. After consuming the composition, the patient experiences a reduction in the production of saliva, as well as an increase in a density of the saliva that is produced. Upon a subsequent coughing event and a subsequent sneezing event, after consuming the composition, the patient expels saliva droplets into the surrounding area that include a weight greater than the aerosolized droplets pre-consumption. The heavier saliva droplets fall to a ground surface at an increased rate of acceleration as compared with the saliva prior to consuming the composition.

### Prophetic Example 6

A 40-year-old patient presents with mucoid congestion of the nasal passage, as well as a cough. The patient experiences coughing and sneezing events resulting in the ejection of mucous particles from the patient's nose and mouth into the surrounding area. The patient wears a face covering during the coughing and sneezing events, such that the patient's nose and mouth are covered during the coughing and sneezing events. The coughing and sneezing events result in aerosolized droplets into the surrounding area, which are concentrating in the area immediately surrounding the patient due to the face covering. The patient receives and consumes a composition includes 2% (w/w) of a thickening agent and 5% (w/w) of a saliva-reduction agent. After consuming the composition, the patient experiences a reduction in the production of saliva, as well as an increase in a density of the saliva that is produced. Upon a subsequent coughing event and a subsequent sneezing event, after consuming the composition, the patient expels saliva droplets into the surrounding area that include a weight greater than the aerosolized droplets pre-consumption. The heavier saliva droplets fall to a ground surface at an increased rate of acceleration as compared with the saliva prior to consuming the composition.

### CONCLUSION

The unmodified saliva particulates carry the most forward momentum, have an upward trajectory with respect to the mouth of the subject, and include noticeably smaller particulates, inferring that the particulates remain suspended in the air surrounding the subject for longer than the other two cases considered (since the unmodified saliva droplets have a very low vertical velocity). The mask case has the largest spread at 105 degrees; however, because the masked droplets have the least forward momentum of the three cases, the droplets does not travel as far along the horizontal axis. However, because the masked particles cover such a wide vertical distance, being about 7 times higher than the modified saliva case, the smaller particles are likely to remain suspended in air for an extended period of time before settling to a ground surface. The modified saliva case has the narrowest spread, the highest rate of descent, and includes noticeably larger particles. Though the modified saliva droplets include forward momentum, the particulates do not tend to remain suspended or travel much upward distance (most likely caused by the change in the saliva's density). This is noted by the absence of a dense cloud of suspended particles for these cases. This indicates that both small and large particulates have a higher tendency of descension versus suspension and would settle the ground sooner than both the unmodified saliva and the masked droplets test cases.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A consumable composition to increase at least one of a density, a viscosity, and a surface tension of saliva, such that the composition is configured to reduce a transmissibility of airborne pathogens, the consumable composition comprising:
a thickening agent selected from the group consisting of cornstarch, xanthan gum, glucomannan powder, gelatin, peanut flour, agar-agar, arrowroot flour, algin, carrageenan, acacin, gum tragacanth, pectin, and combinations thereof;
a saliva-reduction agent selected from the group consisting of a starch beta blocker, a natural beta blocker, an oil, a natural extract, lemongrass oil, ginger extract, cinnamaldehyde, vanillin, peppermint, and combinations thereof;
a bonding agent; and
a food grade carrier encapsulating the thickening agent, the saliva-reduction agent, and the bonding agent therein, the food grade carrier selected from the group consisting of a cough drop, a dissolvable strip, a gum, and an edible food item.

2. The consumable composition of claim 1, wherein the food grade carrier is the edible food item, wherein the edible food item is a chocolate-based food item.

3. The consumable composition of claim 1, wherein the thickening agent is cornstarch.

4. The consumable composition of claim 3, wherein the saliva-reduction agent is a starch beta blocker.

5. The consumable composition of claim 4, wherein the food grade carrier is a chocolate-based food item.

6. The consumable composition of claim 1, further comprising a palatability agent within the food grade carrier.

7. The consumable composition of claim 6, wherein the palatability agent is selected from the group consisting of honey, cocoa butter, a flavoring oil, a fruit flavoring oil, a fruit flavoring essence, an aldehyde, an ester, and combinations thereof.

8. The consumable composition of claim 6, wherein the palatability agent is present within the composition in an amount of less than 50% (w/w).

9. The consumable composition of claim 1, further comprising a nutritional supplement within the food grade carrier.

10. The consumable composition of claim 9, wherein the nutritional supplement selected from the group consisting of vitamin B, vitamin C, vitamin D3, zinc, N-acetylcysteine, melatonin, and combinations thereof.

11. The consumable composition of claim 1, wherein the thickening agent is present within the composition in an amount of between 2% and 40% (w/w).

12. The consumable composition of claim 1, wherein the saliva-reduction agent is present within the composition in an amount of between 1% and 25% (w/w).

13. The consumable composition of claim 1, wherein the bonding agent is present within the composition in an amount of between 20% and 95% (w/w).

14. A method of reducing a transmissibility of airborne pathogens, the method comprising the steps of:
combining a synergistic composition including a thickening agent, a saliva-reduction agent, and a bonding agent in a food grade carrier that encapsulates the thickening agent, the saliva-reduction agent, and the bonding agent therein;
applying the synergistic composition to a user;
decreasing, via the saliva-reduction agent, saliva production from a salivary gland of the user;
reducing, via the saliva-reduction agent, a transmissibility of airborne pathogens due to the decreased saliva produced by the salivary gland, resulting in fewer exhaled saliva droplets, thereby reducing exhaled saliva droplet transmission;
increasing, via the thickening agent, at least one of a density, a viscosity, and a surface tension of saliva produced by the salivary gland of the user, thereby thickening the produced saliva; and
reducing, via the thickening agent, the transmissibility of airborne pathogens due to the thickened saliva produced by the salivary gland, resulting in exhaled saliva droplets translating toward a ground surface at an increased downward vertical velocity and a decreased horizontal velocity as compared with unmodified saliva droplets, thereby reducing exhaled saliva droplet transmission,
wherein the thickening agent and the saliva-reduction agent act synergistically to reduce saliva production and thicken saliva particles to reduce a suspension time of saliva droplets, thereby synergistically reducing the transmissibility of airborne pathogens.

15. The method of claim 14, wherein the thickening agent is selected from the group consisting of cornstarch, xanthan gum, glucomannan powder, gelatin, peanut flour, agar-agar, arrowroot flour, algin, carrageenan, acacin, gum tragacanth, pectin, and combinations thereof, wherein the thickening agent is present within the composition in an amount of between 2% and 40% (w/w).

16. The method of claim 14, wherein the saliva-reduction agent is selected from the group consisting of a starch beta blocker, a natural beta blocker, an oil, a natural extract, lemongrass oil, ginger extract, cinnamaldehyde, vanillin, peppermint, and combinations thereof, wherein the saliva-reduction agent is present within the composition in an amount of between 1% and 25% (w/w).

17. The method of claim 14, wherein the food grade carrier encapsulating the thickening agent, the saliva-reduction agent, and the bonding agent therein, the food grade carrier selected from the group consisting of a cough drop, a dissolvable strip, a gum, and an edible food item.

18. A synergistic consumable composition to increase at least one of a density, a viscosity, and a surface tension of saliva, such that the composition is configured to reduce a transmissibility of airborne pathogens, the synergistic composition comprising:
a thickening agent selected from the group consisting of cornstarch, xanthan gum, glucomannan powder, gelatin, peanut flour, agar-agar, arrowroot flour, algin, carrageenan, acacin, gum tragacanth, pectin, and combinations thereof, wherein the thickening agent is present within the composition in an amount of between 2% and 40% (w/w);
a saliva-reduction agent selected from the group consisting of a starch beta blocker, a natural beta blocker, an oil, a natural extract, lemongrass oil, ginger extract, cinnamaldehyde, vanillin, peppermint, and combinations thereof, wherein the saliva-reduction agent is present within the composition in an amount of between 1% and 25% (w/w);
a bonding agent, wherein the bonding agent is present within the composition in an amount of between 20% and 95% (w/w); and
a food grade carrier encapsulating the thickening agent, the saliva-reduction agent, and the bonding agent therein, the food grade carrier selected from the group consisting of a cough drop, a dissolvable strip, a gum, and an edible food item,
wherein the thickening agent is configured to interact with a salivary gland of a user to increase at least one of a density, a viscosity, and a surface tension of saliva produced by the salivary gland thereby thickening the produced saliva,
wherein the thickening agent is configured to reduce the transmissibility of airborne pathogens due to the thickened saliva produced by the salivary gland, resulting in exhaled saliva droplets translating toward a ground surface at an increased downward vertical velocity and a decreased horizontal velocity compared with unmodified saliva droplets, thereby reducing exhaled saliva droplet transmission,
wherein the saliva reduction agent is configured to interact with the salivary gland of the user to decrease saliva production, and
wherein the saliva reduction agent is configured to reduce the transmissibility of airborne pathogens due to the decreased saliva produced by the salivary gland, resulting in fewer exhaled saliva droplets, thereby reducing exhaled saliva droplet transmission.

19. The synergistic consumable composition of claim 18, wherein the food grade carrier is the edible food item, wherein the edible food item is a chocolate-based food item.

20. The synergistic consumable composition of claim 18, further comprising a palatability agent within the food grade carrier in an amount of less than 50% (w/w), wherein the palatability agent is selected from the group consisting of honey, cocoa butter, a flavoring oil, a fruit flavoring oil, a fruit flavoring essence, an aldehyde, an ester, and combinations thereof.
